(19) United States
(12) Patent Application Publication
(10) Pub. No.: US 2024/0142684 A1
(43) Pub. Date: May 2, 2024

(54) Color Coatings Having Diamond-Like Carbon Layer

(71) Applicant: Apple Inc., Cupertino, CA (US)

(72) Inventors: Bin Fan, Sunnyvale, CA (US); Brian S. Tryon, Redwood City, CA (US); Xiaofan Niu, San Jose, CA (US); Chia-Yeh Lee, San Jose, CA (US); Frank C. Sit, San Jose, CA (US); Hien Minh H Le, San Jose, CA (US); Justin S. Shi, Santa Clara, CA (US); Shinjita Acharya, Santa Clara, CA (US); Ziqing Duan, San Jose, CA (US)

(21) Appl. No.: 18/487,004

(22) Filed: Oct. 13, 2023

Related U.S. Application Data
(60) Provisional application No. 63/419,613, filed on Oct. 26, 2022.

(51) Int. Cl. G02B 5/28; A61B 5/263; A61B 5/28; A61B 5/318

(52) U.S. Cl. CPC G02B 5/286; A61B 5/263; A61B 5/28; A61B 5/318; A61B 5/742

(57) ABSTRACT

An electronic device such as a wristwatch may be provided with conductive structures. The conductive structures may include a sensor electrode for an electrocardiogram (ECG) sensor. A coating may be disposed on the sensor electrode to reflect particular wavelengths of visible light so that the sensor electrode exhibits a desired color. The coating may include adhesion and transition layers on the sensor electrode, an opaque coloring layer on the adhesion and transition layers, and a thin-film interference filter on the opaque coloring layer. The thin-film interference filter may have an uppermost diamond-like carbon (DLC) layer. The DLC layer may contribute to the color response of the coating while concurrently minimizing noise in ECG waveforms gathered by the ECG sensor using the sensor electrode.

This application claims the benefit of U.S. Provisional Patent Application No. 63/419,613, filed Oct. 26, 2022, which is hereby incorporated by reference herein in its entirety.

### FIELD

This disclosure relates generally to coatings for electronic device structures and, more particularly, to visible-light-reflecting coatings for conductive electronic device structures.

### BACKGROUND

Electronic devices such as cellular telephones, computers, watches, and other devices contain conductive structures such as conductive housing structures. The conductive structures are provided with a coating that reflects particular wavelengths of light so that the conductive components exhibit a desired visible color.

It can be challenging to provide coatings such as these with a desired color brightness. In addition, if care is not taken, the coatings may exhibit unsatisfactory optical performance across different operating environments and conductive structure geometries and can undesirably deteriorate the performance of other device components such as sensors.

### SUMMARY

An electronic device may include conductive structures such as a sensor electrode for an electrocardiogram (ECG) sensor. The ECG sensor may gather ECG data using the sensor electrode. A visible-light-reflecting coating may be disposed on the sensor electrode.

The visible-light-reflecting coating may have adhesion and transition layers, an opaque coloring layer on the adhesion and transition layers, and a thin-film interference filter on the opaque coloring layer. The thin-film interference filter may have an uppermost layer that includes diamond-like carbon (DLC). The thin-film interference filter may have a lowermost CrC layer, for example. The opaque coloring layer may include TiCN or TiCrCN, as two examples.

The thin-film interference filter and the DLC layer may help to impart the coating and thus the sensor electrode with a light yellow or gold color. At the same time, the DLC layer may mitigate Faraday current between the sensor electrode and skin, may reduce double layer capacitance between the sensor electrode and skin, and may reduce the water cone angle of moisture on the coating. This may serve to minimize noise in the ECG data gathered using the sensor electrode, helping to ensure that accurate ECG data is gathered over time.

An aspect of the disclosure provides an apparatus. The apparatus can include a conductive substrate. The apparatus can include a coating on the conductive substrate and having a color. The coating can include adhesion and transition layers. The coating can include a thin-film interference filter on the adhesion and transition layers, wherein the thin-film interference filter comprises a diamond-like carbon (DLC) layer.

Another aspect of the disclosure provides an apparatus. The apparatus can include a conductive substrate. The apparatus can include a coating on the conductive substrate and having a color. The coating can include adhesion and transition layers. The coating can include an opaque layer on the adhesion and transition layers. The coating can include a two-layer thin-film interference filter on the opaque layer, the two-layer thin-film interference filter having an uppermost layer comprising diamond-like carbon (DLC).

Yet another aspect of the disclosure provides an electronic device. The electronic device can include a housing. The electronic device can include a display mounted to the housing. The electronic device can include a sensor electrode on the housing. The electronic device can include circuitry configured to gather sensor data using the sensor electrode. The electronic device can include a coating on the sensor electrode and having a color, wherein the coating comprises a diamond-like carbon (DLC) layer that forms part of a thin-film interference filter.

### BRIEF DESCRIPTION OF THE DRAWINGS

FIG. 1 is a perspective view of an illustrative electronic device of the type that may be provided with conductive structures and visible-light-reflecting coatings in accordance with some embodiments.

FIG. 2 is cross-sectional side view of an illustrative electronic device having conductive structures that may be provided with visible-light-reflecting coatings in accordance with some embodiments.

FIG. 3 is an exploded cross-sectional side view of an illustrative conductive housing sidewall that may be provided with a visible-light-reflecting coating in accordance with some embodiments.

FIG. 4 is a cross-sectional side view of an illustrative visible-light-reflecting coating having a multi-layer interference film that includes an uppermost diamond-like carbon (DLC) layer in accordance with some embodiments.

FIG. 5 is a cross-sectional side view of illustrative layers in a visible-light-reflecting coating having a multi-layer interference film with an uppermost DLC layer in accordance with some embodiments.

FIG. 6 is a plot of an L* b* color space for illustrative visible-light-reflecting coatings of the type shown in FIGS. 4 and 5 in accordance with some embodiments.

FIG. 7 is a diagram showing how an illustrative visible-light-reflecting coating of the type shown in FIGS. 4 and 5 may be disposed on a sensor electrode in accordance with some embodiments.

FIG. 8 is a diagram showing how an illustrative visible-light-reflecting coating of the type shown in FIGS. 4 and 5 may reduce noise in sensor data gathered by an underlying sensor electrode in accordance with some embodiments.

FIG. 9 is a ternary phase diagram showing how an illustrative DLC layer may be provided with an sp³-rich composition to minimize noise in sensor data gathered by an underlying sensor electrode in accordance with some embodiments.

FIG. 10 is a cross-sectional side view of illustrative layers in a visible-light-reflecting coating having a three-layer interference film with an uppermost DLC layer in accordance with some embodiments.

### DETAILED DESCRIPTION

Electronic devices and other items may be provided with conductive structures. The conductive structures may include a sensor electrode for an electrocardiogram (ECG) sensor. A coating may be formed on the sensor electrode to reflect particular wavelengths of visible light so that the sensor electrode exhibits a desired color. The coating may include adhesion and transition layers on the sensor electrode, an opaque coloring layer on the adhesion and transition layers, and a thin-film interference filter on the opaque coloring layer. The thin-film interference filter may have an uppermost diamond-like carbon (DLC) layer. The DLC layer may contribute to the color response of the coating, helping to configure the coating to exhibit a robust light yellow or gold color. At the same time, the DLC layer may serve to minimize noise in ECG waveforms gathered by the ECG sensor using the sensor electrode.

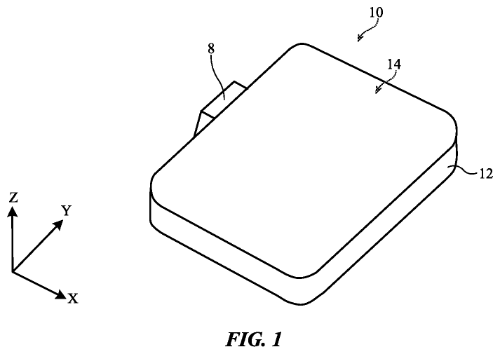

An illustrative electronic device of the type that may be provided with conductive structures and visible-light-reflecting coatings is shown in FIG. 1. Electronic device 10 of FIG. 1 may be a computing device such as a laptop computer, a computer monitor containing an embedded computer, a tablet computer, a cellular telephone, a media player, or other handheld or portable electronic device, a smaller device such as a wristwatch device (e.g., a watch with a wrist strap), a pendant device, a headphone or earpiece device, a device embedded in eyeglasses or other equipment worn on a user's head (e.g., a head mounted device), or other wearable or miniature device, a television, a computer display that does not contain an embedded computer, a gaming device, a navigation device, an embedded system such as a system in which electronic equipment with a display is mounted in a kiosk or automobile, a wireless base station, a home entertainment system, a wireless speaker device, a wireless access point, equipment that implements the functionality of two or more of these devices, or other electronic equipment. In the illustrative configuration of FIG. 1, device 10 is a portable device such as a wristwatch (e.g., a smart watch). Other configurations may be used for device 10 if desired. The example of FIG. 1 is merely illustrative.

In the example of FIG. 1, device 10 includes a display such as display 14. Display 14 may be mounted in a housing such as housing 12. Housing 12, which may sometimes be referred to as an enclosure or case, may be formed of plastic, glass, ceramics, fiber composites, metal (e.g., stainless steel, aluminum, etc.), other suitable materials, or a combination of any two or more of these materials. Housing 12 may be formed using a unibody configuration in which some or all of housing 12 is machined or molded as a single structure or may be formed using multiple structures (e.g., an internal frame structure, one or more structures that form exterior housing surfaces, etc.). Housing 12 may have metal sidewalls or sidewalls formed from other materials. Examples of metal materials that may be used for forming housing 12 include stainless steel, aluminum, silver, gold, titanium, metal alloys, or any other desired conductive material.

Display 14 may be formed at (e.g., mounted on) the front side (face) of device 10. Housing 12 may have a rear housing wall on the rear side (face) of device 10 that opposes the front face of device 10. Conductive housing sidewalls in housing 12 may surround the periphery of device 10. The rear housing wall of housing 12 may be formed from conductive materials and/or dielectric materials.

The rear housing wall of housing 12 and/or display 14 may extend across some or all of the length (e.g., parallel to the X-axis of FIG. 1) and width (e.g., parallel to the Y-axis) of device 10. Conductive sidewalls of housing 12 may extend across some or all of the height of device 10 (e.g., parallel to Z-axis).

Display 14 may be a touch screen display that incorporates a layer of conductive capacitive touch sensor electrodes or other touch sensor components (e.g., resistive touch sensor components, acoustic touch sensor components, force-based touch sensor components, light-based touch sensor components, etc.) or may be a display that is not touch-sensitive. Capacitive touch screen electrodes may be formed from an array of indium tin oxide pads or other transparent conductive structures.

Display 14 may include an array of display pixels formed from liquid crystal display (LCD) components, an array of electrophoretic display pixels, an array of plasma display pixels, an array of organic light-emitting diode (OLED) display pixels, an array of electrowetting display pixels, or display pixels based on other display technologies.

Display 14 may be protected using a display cover layer. The display cover layer may be formed from a transparent material such as glass, plastic, sapphire or other crystalline dielectric materials, ceramic, or other clear materials. The display cover layer may extend across substantially all of the length and width of device 10, for example.

Device 10 may include buttons such as button 8. There may be any suitable number of buttons in device 10 (e.g., a single button, more than one button, two or more buttons, five or more buttons, etc.). Buttons such as button 8 may be located in openings in housing 12 or in an opening in display 14 (as examples). Buttons such as button 8 may be rotary buttons, sliding buttons, buttons that are actuated by pressing on a movable button member, buttons that can receive a user input by being rotated, touched, and/or pressed, etc. Button members for buttons such as button 8 may be formed from metal, glass, plastic, or other materials. If desired, a button member for buttons such as button 8 may also form a sensor electrode for one or more sensors in device 10. Button 8 may sometimes be referred to as a crown in scenarios where device 10 is a wristwatch device.

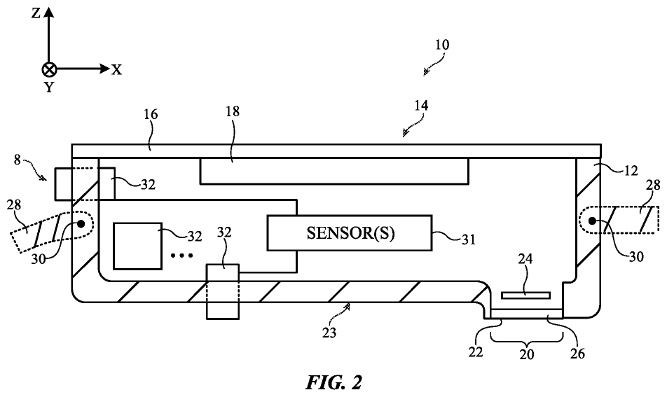

A cross-sectional side view of device 10 in an illustrative configuration in which display 14 has a display cover layer is shown in FIG. 2. As shown in FIG. 2, display 14 may have one or more display layers that form pixel array 18. During operation, pixel array 18 forms images for a user in an active area of display 14. Display 14 may also have inactive areas (e.g., areas along the border of pixel array 18) that are free of pixels and that do not produce images. Display cover layer 16 of FIG. 2 overlaps pixel array 18 in the active area and overlaps electrical components in device 10.

Display cover layer 16 may be formed from a transparent material such as glass, plastic, ceramic, or crystalline materials such as sapphire. Illustrative configurations in which a display cover layer and other transparent members in device 10 (e.g., windows for cameras and other light-based devices that are formed in openings in housing 12) are formed from a hard transparent crystalline material such as sapphire (sometimes referred to as corundum or crystalline aluminum oxide) may sometimes be described herein as an example. Sapphire makes a satisfactory material for display cover layers and windows due to its hardness (9 Mohs). In general, however, these transparent members may be formed from any suitable material.

Display cover layer 16 for display 14 may be planar or curved and may have a rectangular outline, a circular outline, or outlines of other shapes. If desired, openings may be formed in the display cover layer. For example, an opening may be formed in the display cover layer to accommodate a button such as button 8, a speaker port, or other component. Openings may be formed in housing 12 to form communications or data ports (e.g., an audio jack port, a digital data port, a port for a subscriber identity module (SIM) card, etc.), to form openings for buttons, or to form audio ports (e.g., openings for speakers and/or microphones).

Device 10 may, if desired, be coupled to a strap such as strap 28 (e.g., in scenarios where device 10 is a wristwatch device). Strap 28 may be used to hold device 10 against a user's wrist (as an example). Strap 28 may sometimes be referred to herein as wrist strap 28. In the example of FIG. 2, wrist strap 28 is connected to attachment structures 30 in housing 12 at opposing sides of device 10. Attachment structures 30 may include lugs, pins, springs, clips, brackets, and/or other attachment mechanisms that configure housing 12 to receive wrist strap 28. Configurations that do not include straps may also be used for device 10.

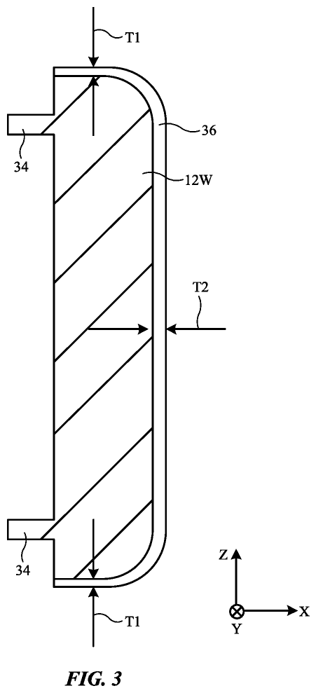

If desired, light-based components such as light-based components 24 may be mounted in alignment with an opening 20 in housing 12. Opening 20 may be circular, may be rectangular, may have an oval shape, may have a triangular shape, may have other shapes with straight and/or curved edges, or may have other suitable shapes (outlines when viewed from above). Window member 26 may be mounted in window opening 20 of housing 12 so that window member 26 overlaps component 18. A gasket, bezel, adhesive, screws, or other fastening mechanisms may be used in attaching window member 26 to housing 12. Surface 22 of window member 26 may lie flush with exterior surface 23 of housing 12, may be recessed below exterior surface 23, or may, as shown in FIG. 3, be proud of exterior surface 23 (e.g., surface 22 may lie in a plane that protrudes away from surface 23 in the −Z direction). In other words, window member 26 may be mounted to a protruding portion of housing 12. Surface 23 may, for example, form the rear face of housing 12.

Conductive structures in device 10 may be provided with a visible-light-reflecting coating that reflects certain wavelengths of light so that the conductive structures exhibit a desired aesthetic appearance (e.g., a desired color, reflectivity, etc.). The conductive structures in device 10 may include, for example, conductive portions of housing 12 (e.g., conductive sidewalls for device 10, a conductive rear wall for device 10, a protruding portion of housing 12 used to mount window member 26, etc.), attachment structures 30, conductive portions of wrist strap 28, a conductive mesh, conductive components 32, and/or any other desired conductive structures on device 10.

Conductive components 32 may include internal components (e.g., internal housing members, a conductive frame, a conductive chassis, a conductive support plate, conductive brackets, conductive clips, conductive springs, input-output components or devices, etc.), components that lie both at the interior and exterior of device 10 (e.g., a conductive SIM card tray or SIM card port, a data port, a microphone port, a speaker port, a conductive button member for button 8, etc.), components that are mounted at the exterior of device 10 (e.g., conductive portions of strap 28 such as a clasp for strap 28), and/or any other desired conductive structures on device 10.

As shown in FIG. 2, device 10 may also include one or more sensors such as sensor 31. Sensor 31 may generate (e.g., gather, sense, or measure) sensor data using one or more conductive components 32. Sensors 31 may include, for example, an optical sensor that gathers optical sensor data using conductive components 32, a touch sensor that detects a user's touch using conductive components 32, a force sensor that detects a force applied to device 10 using conductive components 32, a temperature sensor that gathers temperature data using conductive components 32, or any other desired sensors.

An implementation in which sensor 31 is an electrocardiogram (ECG) sensor that gathers ECG data using conductive components 32 is described herein as an example. The ECG data may include measurements of the electrical activity of a user's heart (e.g., electrical potential) while one or more conductive components 32 are in contact with the user's body. For example, the conductive components 32 that protrude through housing 12 may form sensor electrodes (e.g., ECG sensor electrodes) that measure ECG data while the user is wearing device 10 on their wrist (e.g., sensor 31 may include a first conductive component 32 that forms a first sensor electrode that protrudes through the rear housing wall of device 10 beyond exterior surface 23 opposite display 14 and/or may include a second conductive component 32 that forms a second sensor electrode on or formed from button 8).

FIG. 3 is an exploded cross-sectional side view of a conductive sidewall in device 10 that may be provided with a visible-light-reflecting coating. As shown in FIG. 3, housing 12 may include peripheral conductive housing structures such as conductive sidewall 12W. Conductive sidewall 12W may, for example, run around the lateral periphery of device 10 in the X-Y plane (e.g., conductive sidewall 12W may run around the periphery of display 14 of FIG. 2 and may serve as a conductive bezel for the display).

Conductive sidewall 12W may include one or more ledges 34. Ledges 34 may be used to support a conductive and/or dielectric rear wall for device 10 (e.g., at the rear face of device 10) and/or to support display cover layer 16 of FIG. 2 (e.g., at the front face of device 10). In order to provide conductive sidewall 12W with a desired visible color, a visible-light-reflecting coating such as coating 36 may be deposited onto conductive sidewall 12W (e.g., all of conductive sidewall 12W, the portions of conductive sidewall 12W at the exterior of device 10, etc.). Coating 36 may also be deposited over other conductive structures in device 10 (e.g., conductive components 32 of FIG. 2, other conductive portions of housing 12, etc.).

In practice, the coating may have different thicknesses across its surface area due to changes in the underlying geometry of the conductive structure (e.g., because of coating deposition equipment limitations in depositing uniform coatings across the underlying geometry). For example, coating 36 of FIG. 3 may exhibit a first thickness T1 at the bottom and top edges of conductive sidewall 12W (e.g., where conductive sidewall 12W exhibits a curved three-dimensional shape) but may exhibit a second thickness T2 along the center of conductive sidewall 12W (e.g., where conductive sidewall 12W exhibits a substantially planar shape). Thickness T2 may represent the maximum thickness of coating 36 across its surface area (e.g., 100% thickness). Thickness T1 may be less than thickness T2 (e.g., 30-70% of thickness T2). If care is not taken, variations in thickness along the surface area of coating 36 can undesirably alter the color of visible light reflected by the coating and thus the aesthetic appearance of the underlying conductive structure.

While FIG. 3 shows how the thickness of coating 36 may vary on conductive sidewall 12W, the thickness of coating 36 may also vary across different portions of conductive components 32 of FIG. 2 and/or between different conductive structures on device 10 based on the underlying surface geometry. It may be desirable to provide housing 12 and conductive components 32 (e.g., the sensor electrodes for sensor 31) with a coating 36 that imparts all visible portions of device 10 with a desired uniform color and thus a desired aesthetic appearance despite variations in the thickness the coating as the underlying surface geometry changes across the lateral surface area of the coating.

At the same time, if care is not taken, disposing coating 36 on sensor electrodes for sensor 31 can undesirably deteriorate the quality of the sensor data generated by sensor 31 (e.g., can cause excessive noise in ECG data generated using the sensor electrodes). To mitigate these issues and to configure coating 36 to impart the sensor electrodes for sensor 31 (and the other conductive structures of device 10) with a desired visible color that is invariant as thickness changes and that does not undesirably deteriorate the quality of the sensor data generated by the sensor electrodes of sensor 31, coating 36 may include a multi-layer thin-film interference filter having an uppermost diamond-like carbon (DLC) layer.

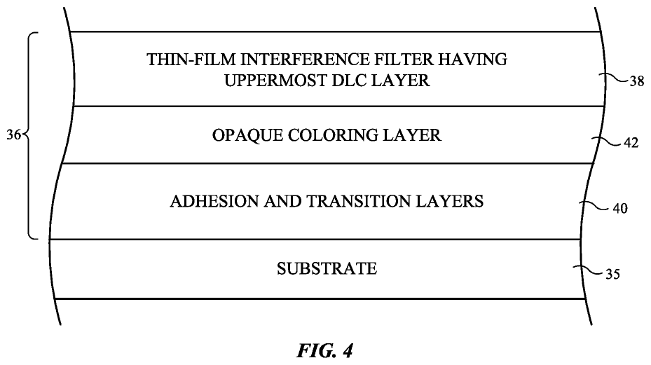

FIG. 4 is a cross-sectional diagram of a visible-light-reflecting coating having a multi-layer thin-film interference filter with an uppermost DLC layer (e.g., for layering onto sensor electrodes for sensor 31 and other conductive structures in device 10). As shown in FIG. 4, a visible-light-reflecting coating such as coating 36 may be disposed (e.g., deposited, layered, formed, etc.) on a conductive substrate such as substrate 35. Substrate 35 may be a conductive structure in device 10 such as a conductive component 32 (e.g., a sensor electrode for sensor 31 that protrudes through the rear wall of device 10 and/or a sensor electrode for sensor 31 that is disposed on or formed from button 8 of FIG. 2), a conductive portion of housing 12 (FIGS. 1 and 2), or conductive sidewall 12W (FIG. 3).

Substrate 35 may be thicker than coating 36. The thickness of substrate 35 may be 0.1 mm to 5 mm, more than 0.3 mm, more than 0.5 mm, between 5 mm and 20 mm, less than 5 mm, less than 2 mm, less than 1.5 mm, or less than 1 mm (as examples). Substrate 35 may include stainless steel, aluminum, titanium, or other metals or alloys. In other suitable arrangements, substrate 35 may be an insulating substrate such as a ceramic substrate, a glass substrate, or substrates formed from other materials.

Coating 36 may include adhesion and transition layers 40 on substrate 35. Coating 36 may include an opaque color layer such as opaque color layer 42 on adhesion and transition layers 40. Coating 36 may include a multi-layer thin-film interference filter such as thin-film interference filter (TFIF) 38 on opaque color layer 42. An optional oleophobic coating or other films, coatings, or layers (e.g., layers that do not substantially contribute to the color response of the coating) may be layered over thin-film interference filter 38 if desired.

Opaque color layer 42 may, for example, have a first lateral surface that directly contacts adhesion and transition layers 40 and may have a second lateral surface opposite the first lateral surface. Thin-film interference filter 38 may, for example, have a third lateral surface that directly contacts the second lateral surface and may have a fourth lateral surface opposite the third lateral surface (e.g., the fourth lateral surface may form an uppermost or outermost layer of coating 36). Thin-film interference filter 38 may include multiple layers (films) stacked on opaque color layer 42. In some implementations, thin-film interference filter 38 may include two stacked layers (films). In other implementations, thin-film interference filter 38 may include three or more stacked layers (films) or a single layer (film).

The layers of coating 36 may be deposited on substrate 35 using any suitable deposition techniques. Examples of techniques that may be used for depositing the layers in coating 36 include physical vapor deposition (PVD) (e.g., evaporation and/or sputtering), cathodic arc deposition, chemical vapor deposition, ion plating, laser ablation, etc. For example, coating 36 may be deposited on substrate 35 in a deposition system having deposition equipment (e.g., a cathode). Substrate 35 may be moved (e.g., rotated) within the deposition system while the deposition equipment (e.g., the cathode) deposits the layers of coating 36. If desired, substrate 35 may be moved/rotated dynamically with respect to speed and/or orientation relative to the deposition equipment (e.g., the cathode) during deposition. This may help provide coating 36 with as uniform a thickness as possible across its area, even in scenarios where substrate 35 has a three-dimensional shape (e.g., minimizing the difference between thicknesses T1 and T2 of FIG. 3).

Thin-film interference filter 38 may be formed from a stack of layers of material such as inorganic and/or organic dielectric layers with different refractive indices. The layers of thin-film interference filter 38 may include one or more layers having higher index of refraction values (sometimes referred to as “high” index values) and one or more layers having lower index of refraction values (sometimes referred to as “low” index values). The layers having higher index of refraction values may be interleaved with the layers having lower index of refraction values if desired.

Incident light may be transmitted through each of the layers in thin-film interference filter 38 while also reflecting off the interfaces between each of the layers, as well as at the interface between the thin-film interference filter and opaque color layer 42 and at the interface between the thin-film interference filter and air. By controlling the thickness and index of refraction (e.g., composition) of each layer in thin-film interference filter 38, the light reflected at each interface may destructively and/or constructively interfere at a selected set of wavelengths such that reflected light that passes out of the thin-film interference filter 38 is perceived by an observer with a desired color and brightness across a corresponding range of viewing angles (angles of incidence, e.g., from 0 to 60 degrees relative to a normal axis of the conductive structure), while also exhibiting a response that is relatively invariant across the lateral area of the coating even when deposited onto an underlying substrate 35 having a three-dimensional (e.g., curved) shape.

Unlike the layers of thin-film interference filter 38, opaque color layer 42 is substantially opaque and does not transmit light incident upon coating 36. On the other hand, opaque color layer 42 may reflect incident light received through thin-film interference filter 38 back towards and through thin-film interference filter 38. The thickness and/or composition of opaque color layer 42 may contribute to the color response of the light upon exiting coating 36 as viewed by a user (e.g., in combination with the interference effects imparted to the transmitted and reflected light by thin-film interference filter 38). Opaque color layer 42 may sometimes also be referred to herein as a non-interference filter layer or an intrinsic color layer.

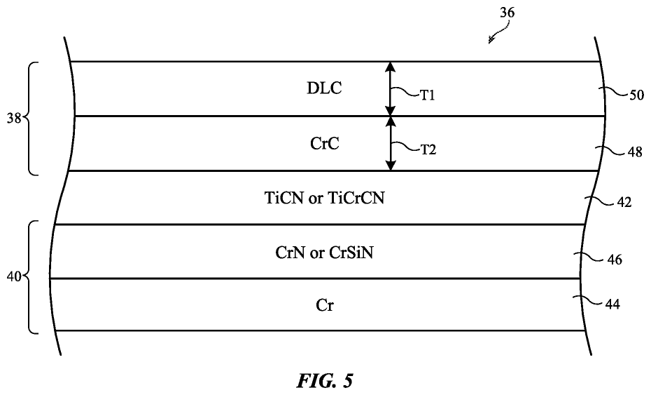

To optimize the performance of sensor 31 (FIG. 2) when coating 36 is layered onto a sensor electrode for the sensor, thin-film interference filter 38 may be provided with an uppermost DLC layer (e.g., the uppermost layer of thin-film interference filter 38 may be a DLC layer). FIG. 5 is a cross-sectional side view showing some illustrative compositions for coating 36 in which thin-film interference filter 38 has an uppermost DLC layer. Substrate 35 has been omitted from FIG. 5 for the sake of clarity.

As shown in FIG. 5, adhesion and transition layers 40 may include a seed (adhesion) layer 44 on substrate 35 and one or more transition layers such as transition layer 46 on seed layer 44. Seed layer 44 may couple substrate 35 to transition layer 46 (e.g., transition layer 46 may be interposed between seed layer 44 and opaque coloring layer 42). In the example of FIG. 5, seed layer 44 is formed from chromium (Cr) and may therefore sometimes be referred to herein as Cr layer 44 or Cr seed layer 44. Transition layer 46 may be formed from chromium nitride (CrN) or chromium silicon nitride (CrSiN) and may therefore sometimes be referred to herein as CrN layer 46, CrSiN layer 46, CrN transition layer 46, or CrSiN transition layer 46. This is merely illustrative. In general, seed layer 44 and/or transition layer 46 may include chromium nitride (CrN), chromium silicon (CrSi), titanium (Ti), chromium silicon nitride (CrSiN), chromium silicon carbonitride (CrSiCN), chromium silicon carbide (CrSiC), chromium carbonitride (CrCN), other metals, metal alloys, and/or other materials.

In the example of FIG. 5, thin-film interference filter 38 is a two-layer interference filter having a first layer 48 and a second layer 50. First layer 48 may be a lowermost (bottom) layer of thin-film interference filter 38 that is layered onto opaque color layer 42. Layer 48 may have thickness T2. Second layer 50 may be an uppermost (top) layer 50 of thin-film interference filter 38 that is layered onto layer 48. Layer 50 may have thickness T1. Thicknesses T1 and T2 and the compositions of layers 48 and 50 may be selected to impart thin-film interference filter 38 with desired interference effects to transmitted and reflected light, thereby configuring coating 36 to reflect visible light with a desired visible color response.

As one example, layer 48 may include chromium carbide (CrC) and may therefore sometimes be referred to herein as CrC layer 44. Layer 50 may include DLC and may therefore sometimes be referred to herein as DLC layer 50. DLC is an amorphous, synthetic, carbon material having a relatively high number of sp³ hybridized carbon atoms (e.g., a ratio of sp³ hybridized atoms relative to other atoms that exceeds a threshold level), which imparts the material with diamond-like properties (e.g., diamond-like hardness, slickness, etc.). The DLC may also include one or more fillers (e.g., non-sp³ hybridized atoms) such as sp² hybridized carbon (e.g., graphite-like carbon) and/or hydrogen. DLC layer 50 may sometimes also be referred to herein as carbon flash layer 50 or carbon flash DLC layer 50.

The example of FIG. 5 is merely illustrative. If desired, thin-film interference filter 38 may include more than two layers (e.g., where DLC layer 50 forms the uppermost layer of the interference filter) or a single layer (e.g., DLC layer 50). Opaque color layer 42 may include titanium carbonitride (TiCN), titanium chromium carbonitride (TiCrCN), or any other desired metals, metal alloys, and/or other materials. Opaque color layer 42 may therefore sometimes be referred to herein as TiCN layer 42 or TiCrCN layer 42.

The composition of DLC layer 50 and CrC layer 48 and thicknesses T1 and T2 may be selected so that coating 36 exhibits a desired a desired color across a predetermined range of angles of incidence. Thicknesses T1 and T2 may both be less than the thickness of opaque color layer 42 (e.g., opaque color layer 42 may be thicker than the sum of thicknesses T1 and T2). Thickness T1 may be less than thickness T2 if desired. Thickness T1 may be, for example, 10-30 nm, 15-25 nm, 18-22 nm, 5-35 nm, 1-50 nm, 1-100 nm, 10-60 nm, 8-23 nm, 18-38 nm, or other thicknesses. Thickness T2 may be, for example, 20-30 nm, 10-40 nm, 22-28 nm, 15-35 nm, 1-50 nm, 1-100 nm, 10-60 nm, 21-45 nm, or other thicknesses.

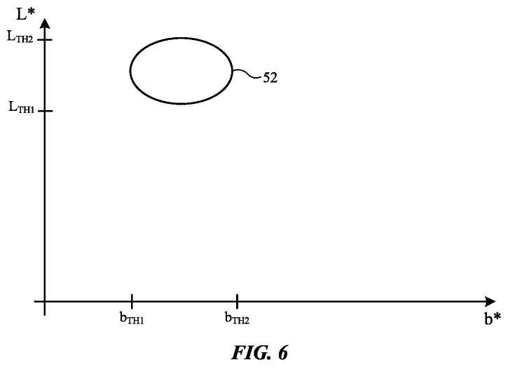

FIG. 6 is a plot of an illustrative color response for coating 36 in L*b* color space. As shown in FIG. 6, thicknesses T1 and T2 and the composition of layers 48 and 50 may be selected to produce interference effects for transmitted and reflected visible light that configure coating 36 to exhibit a color within region 52 (e.g., with an L* value greater than threshold L_{TH1} and less than threshold L_{TH2} and with a b* value greater than threshold b_{TH1} and less than threshold b_{TH2}).

Threshold b_{TH1} may be between 0-10, 5-12, 8-12, 6-12, greater than 0, greater than 4, greater than 5, greater than 6, greater than 8, less than 10, less than 12, less than 15, or other values. Threshold b_{TH2} may be between 12-16, 10-20, greater than 10, greater than 12, greater than 14, greater than 15, less than 15, less than 14, less than 12, less than 20, or other values greater than threshold b_{TH1}. Threshold L_{TH1} may be between 55-70, 60-70, 67-72, greater than 50, greater than 60, greater than 65, greater than 67, greater than 70, less than 70, less than 72, less than 75, or other values. Threshold L_{TH2} may be between 60-75, 71-75, greater than 65, greater than 70, greater than 72, greater than 73, less than 75, less than 80, or other values greater than threshold L_{TH1}.

This may, for example, configure coating 36 to exhibit a light gold, yellow, or champagne color for device 10. In this way, DLC layer 50 may contribute to the visible light interference effects of thin-film interference filter 38 (e.g., may form a part of thin-film interference filter 38), thereby helping to impart coating 36 with the desired visible color response (e.g., helping to place the color response of coating 36 within region 52 of FIG. 6). The example of FIG. 6 is merely illustrative. In practice, region 52 may have other shapes.

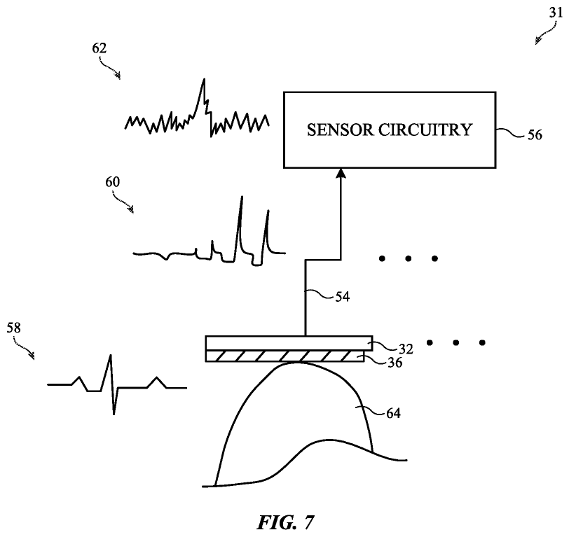

In addition to contributing the visible light interference effects of thin-film interference filter 38, DLC layer 50 may also help to optimize the performance of sensor 31 (FIG. 2) in gathering sensor data (e.g., ECG data). FIG. 7 is a diagram showing how a conductive component 32 provided with coating 36 may be used to gather sensor data such as ECG data (e.g., in an implementation where the coated conductive component 32 is a sensor electrode for sensor 31 of FIG. 2).

As shown in FIG. 7, sensor 31 may include sensor circuitry 56 coupled to one or more conductive components 32 (e.g., one or more sensor electrodes of sensor 31) over one or more signal paths 54. Sensor circuitry 56 may include analog and/or digital circuitry that measures power, voltage, current, phase, and/or any other desired electrical signal characteristics (waveforms) over time (e.g., a voltmeter, ammeter, multimeter, power detector, phase detector, receiver, etc.).

Coating 36 may be disposed on conductive component(s) 32. When a user's body 64 (e.g., skin on the user's wrist, the user's finger, etc.) contacts the sensor electrode (e.g., coating 36), electrical signals (currents) pass from the body 64 to the sensor electrode and to sensor circuitry 56 over signal path(s) 54. The electrical signals may include an intrinsic ECG waveform 58 produced by the electrical activity of the user's heart, which is then received at sensor circuitry 56 as waveform 62.

The sensor electrode is ideally a dry electrode for ECG sensing. Sensor circuitry 56 may receive and measure (e.g., record, sense, generate, etc.) the electrical waveform 62 passed over signal path(s) 54 (e.g., as sensor data or ECG data). Sensor circuitry 56 and/or other control circuitry on device 10 (e.g., one or more processors such as central processing units, microprocessors, application-specific integrated circuits, graphics processing units, etc.) may process waveform 62 to characterize and monitor the user's body and health over time, to detect the occurrence of undesirable cardiac events (e.g., atrial fibrillation), and/or to perform any other desired processing operations.

However, if care is not taken, the sensor electrode may be subject to relatively high levels of noise at the skin-electrode interface (sometimes referred to herein as interface noise). High interface noise can have a negative impact on the accuracy of the ECG detection algorithm implemented by sensor 31, which may compromise clinical accuracy and/or degrade user experience.

For example, a chemical reaction at the skin-electrode interface (e.g., produced by the interaction between the electrical signals and salts on body 64) may produce a spiky Faraday current 60 from intrinsic ECG waveform 58. In addition to the production of spiky Faraday current, noise may be introduced to the electrical signals (e.g., to the waveform 62 as received at sensor circuitry 56) by double layer capacitance between the sensor electrode and body 64 and/or by the surface hydrophobicity of the sensor electrode and coating 36.

These non-idealities may cause the electrical signals to be received and measured (e.g., sensed or gathered) at sensor circuitry 56 with a substantial amount of noise in waveform 62. In other words, if care is not taken, the intrinsically weak ECG signal combined with a spiky Faraday current and/or other interface noise can cause the control circuitry to perform incorrect or inconclusive classification of the user's current ECG waveform.

Double layer capacitance serves as a transducer at the dry skin-electrode interface. The human body contains organic ions such as sodium, potassium, and chlorine ions, whereas electrons conduct the electrical signal on the dry sensor electrode. A double layer capacitance is produced by the electrons and ions at this interface (e.g., due to the presence of sweat between body 64 and the sensor electrode), which converts inorganic ions to electrons that are received at sensor circuitry 56 for recording the physiological signal.

At the same time, when a finger contacts a hard surface, as a result of increased contact area for producing larger friction, the finger becomes moisturized by sweat pores to increase contact area. A hydrophilic interface with the electrode may produce a lower water cone angle for the moisture than a hydrophobic interface, which produces more surface beading for the moisture and thus a higher water cone angle. Lower water cone angles may produce less noise in the electrical signal than higher water cone angles. In other words, a hydrophilic interface can stabilize the double layer capacitance and reduce unintended charge/discharge current at the surface to reduce interface noise.

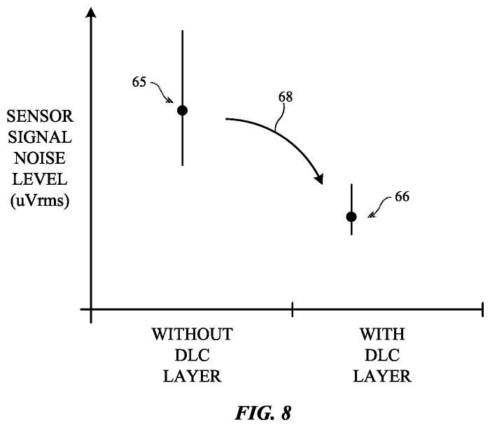

Including DLC layer 50 (FIG. 5) as the uppermost layer in coating 36 may serve to mitigate or reduce this interface noise, thereby minimizing the noise in the waveform 62 received at sensor circuitry 56 (e.g., minimizing the noise in the sensor data gathered by sensor 31). FIG. 8 is a plot showing how including DLC layer 50 (FIG. 5) as the uppermost layer in coating 36 may serve to mitigate or reduce interface noise.

Data 65 of FIG. 8 characterizes the noise of waveform 62 (in uVrms) and thus the sensor data gathered by sensor circuitry 56 when body 64 contacts coating 36 and the sensor electrode(s) (FIG. 7) in implementations where coating 36 does not include DLC layer 50. Data 66 characterizes the noise of waveform 62 and thus the sensor data gathered by sensor circuitry 56 when body 64 contacts coating 36 and the sensor electrode(s) (FIG. 7) in implementations where coating 36 includes DLC layer 50 as the uppermost layer of thin-film interference filter 38. As shown by data 65 and 66, the inclusion of DLC layer 50 serves to significantly reduce noise in the gathered sensor data (as shown by arrow 68). At the same time, DLC layer 50 contributes visible light interference effects to thin-film interference filter 38 that contributes to the overall color response of coating 36 (e.g., within region 52 of FIG. 6).

DLC layer 50 may serve to reduce noise in waveform 62 due to its chemical inertness (e.g., minimizing the production of spiky Faraday current as shown by waveform 60 of FIG. 7), its surface hydrophilicity (e.g., minimizing the water contact angle of moisture on the sensor electrode), and its production of reduced double layer capacitance at the skin-electrode interface. This reduction in noise may maximize the accuracy with which sensor circuitry 56 measures the electrical activity of the user's heart, the clinical accuracy with which certain health events are detected based on the gathered sensor data (e.g., atrial fibrillation events), and the overall user experience with device 10.

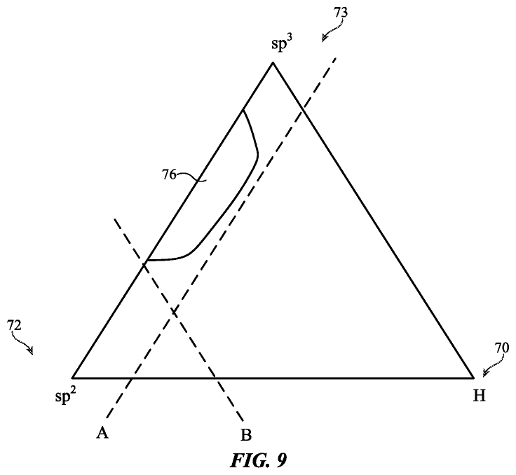

The composition of DLC layer 50 may also be selected to further minimize noise in the gathered sensor data. FIG. 9 is a ternary phase diagram showing how the composition of DLC layer 50 may be selected to further minimize noise in the gathered sensor data. As shown in FIG. 9, the relative composition of DLC layer 50 may be plotted on a ternary phase diagram having a first corner 74 corresponding to pure diamond-like carbon (e.g., pure sp³ hybridized carbon), a second corner 72 corresponding to pure graphite-like carbon (e.g., pure sp² hybridized carbon), and a third corner 70 corresponding to pure hydrocarbons. As hydrocarbons are added to pure graphite-like carbon, the composition (phase) moves away from corner 72 along the horizontal axis towards corner 70, as diamond-like carbon is added to pure graphite-like carbon, the composition moves away from corner 72 along the first diagonal axis towards corner 74, as hydrocarbons are added to pure diamond-like carbon, the composition moves away from corner 74 along the second diagonal axis towards corner 70, etc.

To maximize the sensor data noise reduction produced by DLC layer 50, DLC layer 50 may be provided with a relatively high ratio of sp³ hybridized carbon relative to sp² hybridized carbon and with a relatively high ratio of sp³ hybridized carbon relative to hydrocarbons in DLC layer 50. For example, DLC layer 50 may be provided with a composition that lies within region 76 of the ternary phase diagram of FIG. 9 to minimize noise in the waveform received by sensor circuitry 56. In other words, DLC layer 50 may be provided with a composition having a ratio of sp³ hybridized carbon to sp² hybridized carbon that exceeds threshold level B and having a ratio of sp³ hybridized carbon to hydrocarbons that exceeds threshold level A (e.g., region 76 may lie above thresholds A and B). Threshold A may be 50%, 60%, 70%, 80%, 90%, 95%, more than 50%, more than 40%, more than 60%, more than 70%, more than 80%, more than 90%, more than 95%, or other values. Threshold B may be 50%, 60%, 70%, 80%, 90%, 95%, more than 50%, more than 40%, more than 60%, more than 70%, more than 80%, more than 90%, more than 95%, or other values. Region 76 may have other shapes in practice.

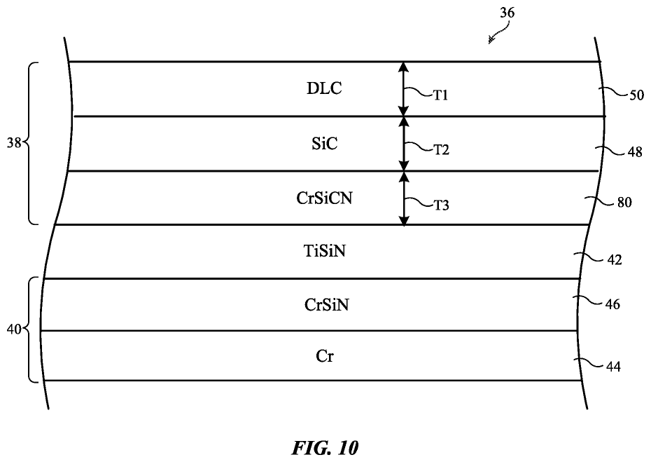

If desired, the thin-film interference filter 38 in coating 36 may be a three-layer thin-film interference filter. FIG. 10 is a cross-sectional side view showing an illustrative composition for coating 36 in an example where thin-film interference filter 38 is a three-layer thin-film interference filter. Substrate 35 has been omitted from FIG. 5 for the sake of clarity.

As shown in FIG. 10, seed layer 44 may be formed from chromium (Cr) and may therefore sometimes be referred to herein as Cr layer 44 or Cr seed layer 44. Transition layer 46 may be formed from chromium silicon nitride (CrSiN) and may therefore sometimes be referred to herein as CrSiN layer 46. This is merely illustrative. In general, seed layer 44 and/or transition layer 46 may include chromium nitride (CrN), chromium silicon (CrSi), titanium (Ti), chromium silicon nitride (CrSiN), chromium silicon carbonitride (CrSiCN), chromium silicon carbide (CrSiC), chromium carbonitride (CrCN), other metals, metal alloys, and/or other materials.

In the example of FIG. 6, thin-film interference filter 38 is a three-layer interference filter having a lowermost layer 80, a middle layer 48, and an uppermost layer 50. Lowermost layer 80 may include chromium silicon carbonitride (CrSiCN) and may therefore sometimes be referred to herein as CrSiCN layer 80. Middle layer 48 may include silicon carbide (SiC) and may therefore sometimes be referred to herein as SiC layer 48. Uppermost layer 50 includes DLC (e.g., as in the implementation of FIG. 5). CrSiCN layer 80 may have thickness T3. Opaque color layer 42 may include titanium silicon nitride (TiSiN) and may therefore sometimes be referred to herein as TiSiN layer 42.

The example of FIG. 10 is merely illustrative. If desired, thin-film interference filter 38 may include more than three layers (e.g., where DLC layer 50 forms the uppermost layer of the interference filter). Opaque color layer 42 may include titanium carbonitride (TiCN), titanium chromium carbonitride (TiCrCN), or any other desired metals, metal alloys, and/or other materials.

The composition of DLC layer 50, SiC layer 48, and CrSiCN layer 80 and thicknesses T1-T3 may be selected so that coating 36 exhibits a desired a desired color across a predetermined range of angles of incidence. As an example, at a location of peak or nominal coating thickness, thickness T1 may be 10-20 nm, 5-25 nm, 1-30 nm, 10-15 nm, 5-17 nm, 2-28 nm, greater than 10 nm, greater than 5 nm, less than 20 nm, less than 50 nm, or other thicknesses. Thickness T2 may be 20-30 nm, 10-40 nm, 5-50 nm, 25-35 nm, 6-60 nm, greater than 20 nm, greater than 10 nm, less than 30 nm, less than 50 nm, or other thicknesses. Thickness T3 may be 80-90 nm, 70-100 nm, 50-120 nm, 25-95 nm, 60-160 nm, greater than 80 nm, greater than 60 nm, greater than 50 nm, less than 100 nm, less than 150 nm, or other thicknesses.

Opaque color layer 42 may have an atomic percentage of titanium atoms of 40-50%, 30-60%, 20-70%, greater than 40%, greater than 30%, less than 50%, less than 60%, or other values. Opaque color layer 42 may have an atomic percentage of silicon atoms of 1-10%, 5-12%, 3-15%, greater than 5%, greater than 1%, less than 10%, less than 20%, or other values. Opaque color layer 42 may have an atomic percentage of nitrogen atoms of 40-50%, 30-60%, 45-55%, greater than 40%, greater than 30%, less than 50%, less than 70%, or other values.

CrSiCN layer 80 may have an atomic percentage of chromium atoms of 1-5%, 1-10%, 2-8%, greater than 2%, greater than 1%, less than 5%, less than 10%, less than 20%, or other values. CrSiCN layer 80 may have an atomic percentage of silicon atoms of 10-20%, 5-30%, 2-40%, greater than 10%, greater than 5%, less than 20%, less than 30%, less than 40%, or other values. CrSiCN layer 80 may have an atomic percentage of nitrogen atoms of 30-40%, 20-50%, 25-45%, greater than 30%, greater than 25%, less than 40%, less than 45%, less than 50%, or other values. The remaining atomic percentage of CrSiCN layer 80 may be carbon atoms.

SiC layer 48 may have an atomic percentage of silicon atoms of 20-30%, 10-40%, 12-38%, greater than 12%, greater than 10%, less than 25%, less than 30%, less than 40%, or other values. The remaining atomic percentage of SiC layer 80 may be carbon atoms. Coating 36 of FIG. 10 may exhibit a light or dusty pink color. For example, at a location of peak coating thickness and a viewing angle of zero degrees relative to the normal axis of the coating, coating 36 of FIG. 10 may have an L* value of 60-80, 65-75, 68-72, 50-90, greater than 65, greater than 60, greater than 55, less than 75, less than 80, or other values, may have an a* value of 0-5, 0-10, −5-15, greater than 4, greater than 1, greater than 0, greater than −5, less than 5, less than 10, less than 15, or other values, and may have a b* value of 0-5, 0-10, −5-15, greater than 3, greater than 1, greater than 0, greater than −5, less than 5, less than 10, less than 15, or other values.

Device 10 may gather and/or use personally identifiable information. It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

The foregoing is merely illustrative and various modifications can be made to the described embodiments. The foregoing embodiments may be implemented individually or in any combination.

What is claimed is:

1. Apparatus comprising:
a conductive substrate; and
a coating on the conductive substrate and having a color, the coating comprising:
adhesion and transition layers, and
a thin-film interference filter on the adhesion and transition layers, wherein the thin-film interference filter comprises a diamond-like carbon (DLC) layer.

2. The apparatus of claim 1, wherein the DLC layer is an uppermost layer of the thin-film interference filter.

3. The apparatus of claim 2, wherein the coating further comprises an opaque layer between the thin-film interference filter and the adhesion and transition layers.

4. The apparatus of claim 3, wherein the thin-film interference filter comprises an additional layer between the DLC layer and the opaque layer.

5. The apparatus of claim 4, wherein the additional layer comprises chromium carbide (CrC).

6. The apparatus of claim 5, wherein the additional layer forms a bottom-most layer of the thin-film interference filter.

7. The apparatus of claim 6, wherein the thin-film interference filter is a two-layer thin-film interference filter.

8. The apparatus of claim 1, wherein the coating further comprises an opaque layer between the thin-film interference filter and the adhesion and transition layers.

9. The apparatus of claim 8, wherein the opaque layer comprises titanium carbonitride (TiCN) or titanium chromium carbonitride (TiCrCN).

10. The apparatus of claim 1, wherein the thin-film interference filter is a three-layer thin-film interference filter, the DLC layer forms an uppermost layer of the three-layer thin-film interference filter, the three-layer thin-film interference filter includes a CrSiCN layer, and the three-layer thin-film interference filter includes a SiC layer between the CrSiCN layer and the DLC layer.

11. The apparatus of claim 1, wherein the coating has an L* value greater than 50 and has a b* value greater than 5.

12. The apparatus of claim 1, wherein the conductive substrate comprises a sensor electrode.

13. Apparatus comprising:
a conductive substrate; and
a coating on the conductive substrate and having a color, the coating comprising:
adhesion and transition layers,
an opaque layer on the adhesion and transition layers, and
a two-layer thin-film interference filter on the opaque layer, the two-layer thin-film interference filter having an uppermost layer comprising diamond-like carbon (DLC).

14. The apparatus of claim 13, wherein the two-layer thin-film interference filter has a lowermost layer that contacts the uppermost layer and that comprises chromium carbide (CrC).

15. The apparatus of claim 14, wherein the opaque layer comprises titanium carbonitride (TiCN) or titanium chromium carbonitride (TiCrCN).

16. The apparatus of claim 15, wherein the adhesion and transition layers comprise a chromium (Cr) seed layer and a transition layer on the Cr seed layer, the transition layer comprising chromium nitride (CrN) or chromium silicon nitride (CrSiN).

17. The apparatus of claim 13, further comprising:
a sensor configured to perform electrocardiogram (ECG) measurements using the conductive structure, wherein the DLC has a ratio of sp³ hybridized carbon to sp² hybridized carbon that exceeds a first threshold value and has a ratio of sp³ hybridized carbon to hydrocarbons that exceeds a second threshold value.

18. An electronic device comprising:
a housing;
a display mounted to the housing;
a sensor electrode on the housing;
circuitry configured to gather sensor data using the sensor electrode; and
a coating on the sensor electrode and having a color, wherein the coating comprises a diamond-like carbon (DLC) layer that forms part of a thin-film interference filter.

19. The electronic device of claim 18, the coating further comprising:
adhesion and transition layers on the sensor electrode; and
an opaque layer on the adhesion and transition layers, wherein the thin-film interference filter has a lowermost layer that contacts the opaque layer, the DLC layer is an uppermost layer of the thin-film interference filter, and the thin-film interference filter has a SiC layer between the lowermost layer and the DLC layer.

20. The electronic device of claim 18, wherein the circuitry is configured to gather electrocardiogram (ECG) data using the sensor electrode and the DLC layer has a ratio of sp³ hybridized carbon to sp² hybridized carbon that exceeds a threshold value.
